(19) United States
(12) Patent Application Publication
(10) Pub. No.: US 2025/0382674 A1
(43) Pub. Date: Dec. 18, 2025

(54) METHOD FOR DETECTING CANCER SUSCEPTIBILITY, EARLY DETECTION AND PREDICTING CANCER BEHAVIOUR

(71) Applicant: Raman Govindarajan, Bangalore (IN)

(72) Inventor: Raman Govindarajan, Bangalore (IN)

(21) Appl. No.: 18/878,672

(22) PCT Filed: Jun. 26, 2023
(86) PCT No.: PCT/IN2023/050619

§ 371 Date: Dec. 24, 2024

(30) Foreign Application Priority Data
Jun. 29, 2022 (IN) 202241037245

(51) Int. Cl. C12Q 1/6886

(52) U.S. Cl. CPC C12Q 1/6886; C12Q 2600/106; C12Q 2600/156

(57) ABSTRACT

A method for detecting cancer susceptibility, early detection and predicting cancer behaviour by using specific markers and parameters described below: screening persons with high risk of getting/having a type of cancer; identifying CNV/SNP/MNP/Indel loci and/or CNV/SNP/MNP/Indel mutations specific to a cancer/cancer stage and common in the buffy coat and tumor of cancer patients/stage of cancer; predicting mutations that will occur when metastasis or recurrence of cancer/tumor occurs by identifying CNV/Indel/SNP/MNP loci and/or Indel/SNP/CNV/MNP mutations in buffy coat and tumor by analysis of defined and timed samples of buffy coat, primary, secondary/recurrent tumor; determining loci and mutations indicative of the tumor behaviour, selected from responsiveness to treatment, toxicity of therapies, dose optimization, minimal residual disease; indicative of progression of disease, timing of recurrence and determining pathways responsible for various behaviours.

### FIELD OF INVENTION

The invention relates to a method for detecting cancer susceptibility, early detection and predicting cancer behaviour by using specific markers and parameters. A number of different steps are involved in the method. The invention is also a method to identify genes and pathways responsible for the various cancer behaviors and to aid rational choice of drugs or combinations to overcome cancer exhibiting various behaviors, personalized for each patient based on the methods described.

### BACKGROUND OF THE INVENTION

Cancer is accepted as a disease of many mutations in the DNA of cells exhibiting “cancerous” behaviour. Mutation is defined as changes in the genetic sequence of a gene or any DNA segment or fragment. Mutations bring out diversity and abnormality in any organism. Mutations can be pathogenic by deleteriously affecting the function of the gene or its protein product by changing its amino acid sequence or expression levels or may be completely harmless. Gene-based Mutations in diseases can be classified as Point mutations (single nucleotide change); MNP's (multiple nucleotide polymorphism), Duplication, Insertion, Deletion. Insertion and/or deletion mutations are referred to as INDELS and include events less than 1 kb in length. Genetic polymorphisms in the human genome is mostly attributed to Copy number variations (CNV's)/SNPs (single nucleotide polymorphism/MNP's) and INDELS, and these mutations are highly associated with multiple human diseases including cancers.

Cancer is unchecked cell growth leading to clonal proliferation of cancerous cells. There can be various triggers for cancer including genetic disorders, viral infections, carcinogen/hormone/environment induced, poor DNA repair mechanisms, poor function of tumor suppressor genes etc. Ultimately all these trigger change in the genetic composition, i.e., mutations, leading to uncontrolled cell division leading to an overgrown group of cells called a tumor, and the spread of tumor cells throughout the body to form new tumors, a process called metastasis. Cancer is a heterogenous disease i.e. each group of cells in a cancer could be different in some way from its neighboring group. Although “clonal expansion” is known to occur and clones of cells (with identical genomes and behaviours) have been documented at a genetic level, several clones have been shown to exist in a given cancer giving groups of cells different properties. Certain mutations have been termed “driver mutations” signifying their importance in cancer development and progression. Targeting of such “driver mutations” has seen considerable success in temporarily “curing” certain cancers. However, it has been repeatedly observed that when one pathway/gene is suppressed with a therapy agent, alternative escape pathways emerge that causes the cancer to recur or become resistant. It is therefore important to identify mechanisms/pathways of resistance and metastasis. Cancers in some patients recur early and, in some patients, recurrence takes many months/years. This varied phenotypic behaviour seen in cancer as a whole depends on a multitude of pathways that act in unison/parallelly and sequentially for the cancer cell to exhibit these behaviours. It is important to note that all these behaviours may not be seen in all cancer cells but these behaviours are seen during the life span of a cancer implying the changing nature of cancer. Identifying behaviours that predominate at a particular stage in each cancer is important to enable appropriate therapies that are stage relevant. While a large number of pathways have been shown to be associated with such behaviours, it has been impossible to identify these “culprit” pathways and determine their dominance at each stage of progression of a cancer. In summary, it can be said that multiple pathways are involved in cancer cell behavior. Hence, cancer detection methods that rely solely on single/few genes and pathways are inadequate. Our invention is a step in the direction of identifying dominant pathways in cancer and the method can be used to enable screening, early detection, predicting response to therapy, monitoring response to therapy, dose and toxicity prediction, prediction of timing of recurrence and indeed even predicting pathways that will occur/dominate when recurrence happens. It is important to recognize that our method complements several other methods in identifying pathways in cancer cell behaviour. While we have carried out our method development using exome data from cancer cells and germline cells, this method could be used equally on transcriptomic data, proteomic data, methylome and metabolomic data and other omics data and for other clinical end points in cancer behaviour.

Cancer is emerging as a major health concern in globally. Many cancers arise from various solid organs and are termed solid cancers, whereas cancers of the blood and bone marrow derived cells are termed liquid cancers. Globocan 2020, showed that the incidence of cancer was 19292789 in 2020 with a 5-year prevalence of 50550287 and deaths due to cancer were 9958133, annually. National cancer registry in India has published that cancer burden in India is going to increase from 1.39 million to 1.57 million by 2025. Cancer of breast, cervix uteri, lungs, oral cavity are most common, and incidence of thyroid cancer, and stomach, colon cancer is steadily increasing. It has been established that if detected early, patients may lead an almost normal life span and have an almost normal quality of life. However, most often cancer is advanced when first detected. It is therefore necessary to have a simple preferably non-invasive test that can be applied to a general population at periodic intervals to detect cancer early. Normal method of detecting cancers are: detection of an abnormality in body functions on blood chemistry or hematology tests; detection of a lump/mass on physical examination, detection of a lump or mass on x-ray, CT, MRI examination of the whole body or its parts; symptoms that are indicative of cancers are abnormal/pathologic fractures, bleeding from various orifices, unexplained weight loss, liver or kidney failure, unresponsive pain, severe vomiting and such other symptoms or findings on investigations. These are all late symptoms and most often these indicate late-stage disease that is incurable or even untreatable. Thus, the need of early diagnosis is paramount and critical if cures or prolonged disease-free survival with a good quality of life is to be achieved.

### Below are Some of the Current Medical Needs in Cancer Detection and Optimal Therapy

1. Detection of susceptibility to develop cancer—. A few well-known markers for susceptibility are mutations in BRACA1 and 2, TP53, PTEN, MSH2, MLH1, MSH6, PMS2, EPCAM, APC, RB1, MEN1, RET, VHL. Mutations in these genes predispose individuals to some cancers. However, we do not have such a list for many cancers.
2. Screening method(s) that detect most of the common cancers early and early detection of recurrence/relapse/metastasis).
   Early detection has been the main reason for reduction in breast cancer related mortality. Routine screening based on age and risk profile of patients has been instituted for breast, cervical, colon, lung and prostate cancers—the screening could be physical exam (breast, prostate, cervix), smear and cytology, imaging (CT/MRI/ultrasound/thermal—for breast, cervix and lung cancers, endoscopy for colon and stomach cancers, blood tests for liver, ovary, prostate cancers (AFP, CA 125, PSA), CEA, CA 19.9 (gastrointestinal cancers), detection of HPV (cervical and oral cancers), biomarkers in blood, and exosomes, cytology of secretions and body fluids, circulating tumor cells and circulating tumor derived DNA. Some of these latter are still research tools and have not made it to the clinic for routine use. It is important to emphasize that there is no single test that will detect all/many cancers.
   Recurrence, resistance and metastasis are the major causes of death in cancer. To overcome these later stages, we need to identify these phenotypes at an early stage using molecular level markers. Current methods include monitoring of known markers as mentioned above. However, we do need additional markers that signify resistance/recurrence or metastasis and not just rely on markers for early detection of the primary disease
3. Response to standard of care—a test that will tell the oncologist if the patient will respond to standard of care or if more advanced therapies are required: There is currently no test to determine if a patient will respond to a particular therapy or will progress inexorably to death. While there are molecular markers/mutations that may signify higher response or poorer response rates these are single gene mutations and do not apply to all patients. There is only a statistical probability of response or lack thereof if particular mutations in these genes are detected. Thus, there is a need for a “personalized determination” and categoric (yes/no) classification of responsiveness to named therapy for each patient with cancer. While some of these mutations can apply to other cancers, there is a need for cancer specific definition of prognostic mutations in a given patient of given ethnicity, for a given treatment. While mutations and markers have been collated from various studies, they have been mostly done with random biopsy samples from tumors and not specifically from potentially/actually resistant or potentially/actually metastatic cells. Also, these studies have been done with nucleic acid extracted from a random sample of cells. Furthermore, since these are collated from several studies, they may or may not apply to a given patient. There is a need to develop a method which determines molecular pathways that are responsible for individual patient/cancer behavior in a more personalized manner.
4. Predictive markers are factors that are associated with upfront response or resistance to a particular therapy. Predictive markers are important in oncology as tumors of the same tissue of origin vary widely in their response to most available systemic therapies. Currently recommended oncological predictive markers include both estrogen and progesterone receptors for identifying patients with breast cancers likely to benefit from hormone therapy, HER-2 for the identification of breast cancer patients likely to benefit from trastuzumab, specific K-RAS mutations for the identification of patients with advanced colorectal cancer unlikely to benefit from either cetuximab or panitumumab and specific EGFR mutations for selecting patients with advanced non-small-cell lung cancer for treatment with tyrosine kinase inhibitors such as gefitinib and erlotinib. The availability of predictive markers should increase drug efficacy and decrease toxicity, thus leading to a more personalized approach to cancer treatment. Such markers are available only for some subtypes of some cancers and even where available they are not definitive. There is an acute need for markers of response or lack thereof to a number of chemo and targeted therapies. It has also been observed that just presence of some markers does not guarantee response or lack thereof to therapies targeting such mutations. Additionally, we need methods to monitor response to therapy and determine if indeed the patient is responding, and if there is residual disease after several cycles of therapy.
5. Monitoring of changes in phenotypic behaviour of cancer (stemming from genetic changes in some cancer cells):
   Cancer evolves in a patient during his/her lifetime. Detection of changes in the genetic makeup of tumors can be done by sampling the tumor and analysing its genetic makeup. However, this is not a convenient process as it will involve hospitalization, expense and a certain amount of risk of morbidity and even mortality. There is a need for developing a method which is easy and minimally invasive like from a blood test.
6. Prediction of changes in phenotypic behaviour of cancer-prediction of development of secondaries and recurrences: We are unable to predict (early) the development of recurrences and metastases. Most often their detection is after a new symptoms or lump is detected on follow up examination. These screening methods are both cumbersome and expensive. We need methods that are non-invasive or minimally invasive to detect recurrence and metastasis development early. Methods are also needed to identify patients in whom recurrence will occur early vs those in whom recurrence will occur much later so that appropriate screening and therapy decisions can be made tailored to the patient and his cancer. It is currently impossible to predict what mutations will occur in a tumor as time progresses. There is a need for developing a method to predict mutations that will occur in the future in a given cancer. This would revolutionize cancer diagnosis and therapy
7. Identification of pathways that drive cancer behavior that can be targeted by small or large molecule or other advanced therapies: Routine genomic sequencing of a cancer tissue does not allow one to determine which of the many mutations are responsible for tumor behaviour.
8. Prediction of dose needed for optimal effect with minimal side effects-cancer therapy has always relied on maximal tolerated dose and not on minimal effective dose (as in other therapy of other diseases). There is a need to develop a method which enables determining if a patient has low toxicity or can tolerate higher doses. Such a prediction can enable determining optimal dose to be given to a every patient instead of maximal tolerated dose of drug/therapy.
9. Prediction of toxicity: We are unable to predict those who will develop toxicity (in defined organs) and those who can tolerate higher doses or longer therapy cycles thus improving overall outcome. This capability will promote a personalized regimen construction for each patient.
10. Prediction of residual disease: We need methods to monitor response to therapy and determine if indeed the patient is responding and if there is residual disease after several cycles of therapy
   Present invention not only enables addressing above stated deficiencies in cancer related studies but also enables determining the pathways that are responsible for each behaviour being studied (resistance/response to therapy, progression, change in phenotypic behaviour etc). In carrying out the analysis stream according to the present invention, the practitioner will be able to identify mutations and pathways that are driving such behaviour—this capability is a boon to drug discovery companies who are constantly searching for novel and validated targets to develop therapies.

### Prior Art Related to Blood Sample Based Genomic Cancer Detection (Liquid Biopsy):

Peripheral blood circulating tumor cells (CTCs) have been detected and shown to have prognostic and predictive value in several solid cancers. However, these preliminary efforts have been hampered by two significant limitations: (1) CTC isolation and (2) CTC detection. Collecting CTCs has involved a laborious process that employs multiple antibody binding and magnetic bead sorting steps, requiring expensive reagents and equipment, and ultimately yielding a relatively small population of CTCs, which may vary from sample to sample depending on the expression pattern of cell surface markers used in this method. Nevertheless, regardless of the isolation method, it is difficult to derive accurate diagnostic, prognostic or predictive data from absolute numbers of CTCs because of the relative paucity of these cells in peripheral blood.

US20130040824A1 provides a method of analyzing a biological sample of an organism, including cell-free DNA fragments originating from normal cells and potentially from cells associated with cancer, for imbalances in chromosomal regions arising due to chromosomal deletions or amplifications associated with cancer. In this method specific locus of first haplotype and second haplotype are determined and their nucleic acid sequences are identified to calculate first value from first haplotype and second value from second haplotype. The comparison of first value with second value determines a classification of whether the chromosomal region exhibits a deletion or an amplification. The method also involves calculating a ratio of the first value and the second value to determine a fractional concentration of cancer DNA in the biological sample. The method allows to diagnose or screen a patient for cancer, as well as prognosticate a patient with cancer.

WO2013190441A2 provides a method for detecting cancer or premalignant change in a subject. The method involves observation of frequency of somatic mutations in a biological sample (e.g., plasma or serum) of a subject undergoing screening or monitoring for cancer, when compared with that in the constitutional DNA of the same subject. False positives can be filtered out by requiring any variant locus to have at least a specified number of variant sequence reads (tags), thereby providing a more accurate parameter. Further the citation provides for monitoring a cancer patient following treatment and to see if there is residual tumor or if the tumor has relapsed; and frequency of somatic mutations of subject undergoing treatment and after treatment is estimated. A patient with residual tumor or in whom the tumor has relapsed would have a higher frequency of somatic mutations than one in whom there is no residual tumor or in whom no tumor relapse is observed.

US20190264291A1 describes a method for detecting tumor-derived mutations in cell-free DNA molecules; wherein, the sequences of cell-free DNA molecules from a biological sample of a subject (first sequences) and compared with DNA molecules from a plurality of blood cells of the subject (second sequences), and the tumor-derived mutations in the cell-free DNA molecules are determined by filtering out a portion of the first sequences that are also present in the second sequences. Here, the constitutional DNA is determined using buffy coat DNA,). Single nucleotide variants (SNVs) present in the tumor DNA but not in the buffy coat DNA were mined with a stringent bioinformatics algorithm to detect cancer.

Most of the citations involve removal of mutations found in DNA of buffy coat (considered as constitutional/germline DNA) for normalization. Such normalization may be helpful to determine some specific tumor biomarkers. Screening of such specific markers may happen only after a patient comes for detection of cancer after appearance of at least 1-2 symptoms. However, the need is for an early diagnosis of cancer in high-risk persons using an easy and simultaneously specific method, or method to detect recurrence of cancer after treatment. Further there are no studies/reliable methods to predict if a patient would be responsive to treatment or not. Taking into consideration of drawbacks of the prior art the present invention provides a method to determine susceptibility of a person to cancer, especially in high-risk populations, a method that can be used as a screen for several cancers, to identify early recurrence of cancer, and a method to identify if a patient is responsive or otherwise to treatment and the pathways implicated in such response or lack thereof. The invention also predicts which patients may develop early recurrence vs those who will develop late recurrence. The invention also provides a method to detect residual disease after therapy and to monitor response to therapy. The invention also provides a method to determine which patients will develop toxicity and what doses may be appropriate for a given patient. The invention also provides a method to predict mutations that will happen in cancer over time. And finally, the method provides a means to identify genes/mutations and pathways that are implicated in cancer behaviours indicated above.

### OBJECT(S) OF THE INVENTION

An object of the present invention is to propose a method for detecting cancer susceptibility, early detection and predicting cancer behaviour (using specific markers and parameters), in the general population or especially in high-risk populations for example smokers, patients with family history of cancer, patients who have undergone radiotherapy or chemotherapy for cancer (detection of recurrence and second cancers), and persons with occupational exposure to carcinogens and viruses that can cause cancer.

Still another object of the present invention is to propose a method of screening for many/all solid/liquid cancers for early detection by a minimally invasive blood test.

Yet another object of the present invention is to propose a method for early detection of metastasis or recurrence of cancer in a person after initial treatment and apparent remission by a minimally invasive blood test.

Further object of the present invention is to propose a method to predict the mutations that will occur in such recurrences or metastasis thus providing the oncologist with information to treat the patient in a personalized manner, by a minimally invasive blood test.

Still, a further object of the present invention is to propose a method to predict response to planned therapy and to thus enable the physician to use alternatives if a patient will not respond to a particular therapy.

Yet another object of the present invention is to propose a test that will predict early vs later recurrence of a particular cancer after standard of care therapy.

Still another object of the present invention is to propose a test that will detect residual disease after therapy by minimally invasive blood.

Yet another object of the present invention is to provide a test that will monitor response to therapy by a minimally invasive blood test.

A further object of the present invention is to propose a method to determine the optimal dose of anti-cancer therapy to be given to a particular patient.

Still further object of the invention is to provide guidance as to which patient may exhibit toxicity at lower doses and which patients may tolerate higher doses and longer therapies.

Yet another object of the present invention is to propose a method to identify mutations and pathways that are novel targets/causes of in various stages of cancer including primary recurrent/resistant and metastatic cancer and use of this information for choosing optimal therapies in the clinic or for new indications for known and new drugs.

### SUMMARY OF THE INVENTION

This invention relates to a method for identification of person susceptible to/having cancer by using specific markers and parameters wherein the method comprises of:
carrying out NGS of buffy coat DNA of normal persons and patients with cancer and determining SNV and indel burden in these two groups. Cancer is suspected in a person being screened for cancer if the indel/SNV burden in higher than in normal individuals.

Further by NGS of tumor and buffy coat of persons with cancer (primary or metastatic/recurrent/resistant), (and buffy coat of normal persons), determining/identifying CNV/SNP/MNP/Indel loci and/or CNV/SNP/MNP/Indel mutations specific to a cancer/cancer stage/cancer behaviour, common in the buffy coat and tumor of cancer patients that are not present in normal individuals;

Further by NGS of tumor and buffy coat (sampled at the beginning of therapy and defined time points thereafter) of persons with cancer who are slated to undergo a particular chemo/other radio/targeted/therapy, identifying CNV/SNP/MNP/Indel loci and/or CNV/SNP/MNP/Indel mutations specific to a response phenotype and common in buffy coat and tumor, and using this mutation list to predict response to therapy, toxicity of therapies, detect minimal residual disease; monitoring of response, predicting if recurrence will occur early or late, and indicative of progression of disease.

Similarly, using timed sampling of patient buffy coat and tumor, determining CNV/SNP/MNP/Indel loci and/or CNV/SNP/MNP/Indel mutations that indicate/predict mutations that will occur when metastasis or recurrence of cancer/tumor occurs.

Thus, the present invention relates to a method to determine susceptibility of a person to cancer especially in high-risk populations e.g. smokers, patients with family history of cancer, screening for cancer in the general population and especially those who have undergone radiotherapy or chemotherapy for cancer (for early detection of recurrence/metastasis and second cancers) and persons with occupational exposure to carcinogens and viruses that can cause cancer. The invention also relates to a method to determine nature of mutations and pathways causing recurrence and resistance of cancer to therapies, and responsiveness or otherwise of a patient to known treatments. The invention also provides a method to detect residual disease after therapy and to monitor response to therapies. The invention also provides a method to predict early and late recurrence. The invention also enables to determine doses or dose range appropriate for individual patients and to identify patients who can tolerate higher doses and longer therapies from those patients who will exhibit toxicity early in the therapy protocol. The invention also enables to predict mutations that will happen over time in a given patient and mutations that cause cancer to evolve.

### DETAILED DESCRIPTION OF THE INVENTION

The present invention relates to a method to determine susceptibility of a person to cancer especially in high-risk populations e.g. smokers, patients with family history of cancer, screening of patients for cancer (in the general population) and especially those who have undergone targeted therapy, radiotherapy or chemotherapy for cancer (for detection of recurrence and second cancers) and persons with occupational exposure to carcinogens and viruses that can cause cancer. The invention also relates to determine nature of mutations and pathways causing particular cancer behaviours such as recurrence (including timing of recurrence), metastasis and resistance of cancer, and responsiveness of a patient to known treatments and screening for residual disease and monitoring response to therapy. The invention also claims to help determine dose or dose range appropriate for individual patients and to identify patients who can tolerate higher doses and longer therapies from those patients who will exhibit toxicity early in the therapy protocol. Further, the invention enables to detect mutations and pathways that are driving cancer various behaviors so as to enable targeted therapies to be developed to these “responsible” pathways. In all instances described in this application, determining of Indel/CNV/SNP/MNP loci, number and mutations are determined by standard NGS methods and bioinformatics tools widely available today:

In one of the embodiments of the present invention, the invention provides a method of screening persons with high risk of getting/having a specific types of cancer or in the general population, wherein, the method comprises of the following steps
1. determining Indel and/or CNV/SNP/MNP burden in DNA of buffy coat of cancer patient/person who is being tested for cancer;
2. determining Indel and/or CNV/SNP/MNP burden in DNA of buffy coat of a normal cancer-free persons with respect to the reference genome, and
3. determining the ratio of Indel and/or CNV/SNP/MNP burden in DNA of buffy coat of the subject being tested and that of a normal cancer-free person;
   Wherein, if the ratio of Indel and/or CNV/SNP/MNP burden of the subject is equal or more than 1.39 times for SNP and indel burden is equal or more than 2 times of normal cancer free subjects, then concluding that the subject is susceptible to a cancer or already has a cancer (Example 3 Table 1). Further, a normal cancer-free person selected should preferably belong to same ethnicity/racial background of the subject in consideration.

The method involves estimating the INDEL/SNP/CNV/MNP burden by carrying out exome/whole genome sequencing of the buffy coat DNA of a subject (person being screened or a normal cancer free persons) using standard DNA sequencing methods/NGS. Here the subject is any person who has come to test for the specific cancer for the first time, general population subjected to screening for the cancer or persons belonging to high-risk group for cancer or a person who has had a cancer, has been treated and is in apparent remission. Said method is more precise and has less possibility of false negative results as it does not rely merely on very few known biomarkers. Simple identification of CNV/SNP/MNP/INDEL burden in subject's buffy coat DNA and its comparison with normal cancer-free person's buffy coat DNA helps in determining if the subject is susceptible to or that the person already has cancer. Further, it is an easy and simple method to evaluate a cancer patient who has undergone cancer therapy and is being monitored for recurrence. Regular evaluation of the cancer patient can detect the recurrence of cancer early.

In another embodiment, the invention provides a method to identify CNV/SNP/MNP/Indel loci and/or CNV/SNP/MNP/Indel mutations specific to a cancer that are indicative of susceptibility, presence of primary cancer or progress even beyond initial primary stage, wherein, the method comprises the steps of:
1. obtaining DNA from buffy coat, and malignant tumors/cancer cells of tissues/organs/bone marrow of a patient with a specific cancer (primary or recurrent or metastatic cancer) and sequencing the DNA of both buffy coat and tumor tissue and establishing the CNV/SNP/MNP/INDEL loci and mutations present and common in both samples;
2. obtaining DNA from buffy coat of a normal cancer-free person/parental/sibling buffy coat DNA sequencing the DNA using standard NGS methods and establishing CNV/SNP/MNPs/INDEL loci and mutations in such cancer free individuals with respect to a reference genome;
3. determining CNV/SNP/MNPs/INDEL loci and mutations which are present in the DNA of both buffy coat and tumors of tissues/organs of the patient with specific cancer, but absent from the DNA of buffy coat of normal cancer-free persons/reference genome/parental/sibling buffy coat genome, wherein, said CNV/SNP/MNPs/INDEL loci and mutations indicative of cancer-specific mutations; and creating a database of said cancer-specific CNV/SNP/MNPs/INDEL loci and mutations obtained in step 3.
4. Using this gene list/database to screen for mutations in DNA of buffy coat of subjects desirous of being screened for cancer or screening of patients in remission for evidence of recurrence/metastasis. If carried out serially the method may be used to monitor response to therapy or to estimate residual disease by screening the buffy coat alone. (Example 4, Table 2).

Further, a normal cancer-free persons selected should preferably belong to same ethnicity/racial background of the cancer patient in consideration or the reference genome selected should be from the same ethnicity.

This database compiled in step 4 is useful to identify if a subject has or is susceptible to that specific cancer if the DNA of buffy coat of the subject displays such CNV/SNP/MNPs/INDEL loci and mutations. This does not require any biopsy or other painful invasive techniques. These mutations at these loci (and other mutations at these loci) preexisted in the person and made him/her susceptible to cancer given the combination of external/environmental exposure factors. Thus, this can also be used to screen for cancer (primary and recurrence) using blood buffy coat without the need for a biopsy. This database may also be used to indicate that the cancer has progressed beyond the initial primary stage in view of the fact that the mutations are seen in both the buffy coat and tumor. This fact may also be used by the oncologist to administer more aggressive therapies rather than what is used for a primary localized cancer. This may also be used to detect recurrence of a cancer in apparent remission.

The above method provides a vaster and elaborate method to identify if a subject is susceptible to cancer instead of relying only on few biomarkers as it encompasses the entire exome. Once this process is followed for one cancer it may be repeated for other cancers and the databases for each cancer may be combined to obtain a more comprehensive screening database for multiple cancers. The method may also be used to monitor response to therapy and estimate residual disease.

In yet another embodiment the invention, the invention provides a method to identify loci and/or Indel/SNP/CNV/MNP mutations in chromosomes which indicate recurrence of cancer/tumor and metastasis, wherein, the method comprises the steps of:
1. obtaining DNA from buffy coat and tumor of cancer patients with metastasis or recurrence and identifying CNV/SNP/MNP/Indel loci and CNV/SNP/MNP/Indel mutations common in DNA of buffy coat and secondary or recurrent tumor of cancer patient;
2. obtaining DNA from buffy coat normal cancer-free person/reference genome/parental/sibling DNA, and identifying CNV/SNP/MNP/Indel loci and CNV/SNP/MNP/Indel mutations in DNA of buffy coat of normal cancer-free persons with respect to the reference genome;
3. determining CNV/SNPMNP/Indel loci and CNV/SNP/MNP/Indel mutations which are only present in the DNA of buffy coat of recurrent/metastasis cancer patient, but absent or preferably present in DNA of primary/recurrent/metastatic tumor of cancer patient and absent in DNA of buffy coat of normal person/reference genome/parental/sibling DNA, the said loci and CNV/SNP/MNPs/INDEL mutations indicative of recurrence of cancer/tumor and/or metastasis;
4. creating a database of the specific CNV/SNP/MNP/Indel loci and CNV/SNP/MNP/Indel mutations which are only present in the DNA of buffy coat and may or may not be present in the secondary/recurrent tumor of cancer patient but absent in the buffy coat of normal persons to serve as markers which indicate metastasis or recurrence of cancer. Once this process is followed for one cancer it may be repeated for other cancers and the databases for each cancer may be combined to obtain a more comprehensive screening database for multiple cancers. Screening for such CNV/SNP/MNP/INDEL's may be carried out by various methods such as amplicon sequencing, whole exome sequencing, whole genome sequencing of buffy coat DNA (Example 5, Table 3).

Further, a normal cancer-free persons/reference genome selected should preferably belong to same ethnicity/racial background of the cancer patient in consideration.

Said database is useful to screen for early recurrence or metastasis in patients using buffy coat DNA as the only sample from the patient. Implied in this is the fact that unless the cancer related mutations in the buffy coat are recognized and treated, true cures are unlikely.

This method enables identification of markers of metastasis or recurrence of cancer which serve in early diagnosis of metastasis or recurrence of cancer. These loci and mutations unique to cancer patients buffy coat (not seen in normal person's buffy coat) are loci and mutations therein (and possibly other mutations at these loci) that cause metastasis or recurrence and give new phenotypic features to the cancer.

If a subject with a specific cancer has undergone treatment is evaluated for these markers at an early and later stages of follow-up, one can detect that the subject has metastasis or recurrence of cancer, and accordingly the subject can be advised further diagnostic work up and treatment in advance. This enables early management of the metastasis or recurrence of cancer thereby reducing the chance of loss of life. Further this test may be applied to monitor response to therapy of primary or secondary/recurrent cancers and evaluation of residual disease.

In yet another embodiment the invention, the invention provides a method to identify CNV/Indel/SNP/MNP loci and/or Indel/SNP/CNV/MNP mutations in chromosomes which predict mutations that will occur when metastasis or recurrence of cancer/tumor occurs, wherein, the method comprises the steps of:
1. obtaining DNA from buffy coat and tumor when a patient has the primary cancer—T1, and identifying CNV/SNPMNP/Indel loci and CNV/SNP/MNP/Indel mutations common in DNA of buffy coat and tumors of cancer patients;
2. obtaining DNA of buffy coat and tumor of cancer patient with recurrence/metastasis of tumor after initial therapy (T2) or after more than one round of therapy (T3-Tn) leading to remissions (partial response or full response) and identifying CNV/SNP/MNP/Indel loci and CNV/SNP/MNP/Indel mutations in DNA of buffy coat and/or recurrent/metastatic tumor of patient;
3. determining CNV/SNP/MNP/Indel loci and CNV/SNP/MNP/Indel mutations which are only present and common in the DNA of buffy coat and/or tumor of T2 and/or T3 and are present in the DNA of buffy coat at T1, but not in tumor at T1 to establish a loci and mutations indicative/predictive of mutations in recurrence or metastasis, and creating a database of said CNV/SNP/MNP/Indel loci and CNV/SNP/MNP/Indel mutations;
4. determining CNV/SNP/MNP/Indel loci and CNV/SNP/MNP/Indel mutations which are only present and common in the DNA of buffy coat at T1 and/or secondary/recurrent tumor (T2 or T3) of cancer patient, but absent in DNA of tumor of T1 and DNA of buffy coat of normal person to serve as markers which indicate mutations that may arise at T2 or T3 or Tn as the cancer evolves, and creating a database of said CNV/SNP/MNP/Indel loci and CNV/SNP/MNP/Indel mutations (Example 5, Tables 4,5).

This database is useful to screen for early recurrence or metastasis in patients and determine pathways and mutations that will occur at a future point in time and that can be addressed by specific drugs thus allowing the oncologist to plan in advance for specific therapies.

In yet another embodiment the invention, the invention provides a method to determine mutations indicative of responsiveness to treatment, wherein, the method comprises steps of:
1. Obtaining DNA from buffy coat and tumor of cancer patients belonging to groups that are classified as responder, partial responder or poor responder/progressive disease (RECIST criteria) or any other mode of classification of response (including by pathology, imaging, biomarker driven and other such methods) at T1 before starting any treatment, and identifying CNV/SNP/MNP/Indel loci and CNV/SNP/MNP/Indel mutations common in DNA of tumor at T1 and in buffy coat of such patients.
2. obtaining DNA from buffy coat normal cancer-free person/reference genome/parental/sibling buffy coat genome, identifying CNV/SNP/MNP/Indel loci and CNV/SNPMNP/Indel mutations in DNA of buffy coat of normal cancer-free person/parental/sibling DNA;
3. determining CNV/SNP/MNP/Indel loci and CNV/SNP/MNP/Indel mutations which are present commonly in the DNA of tumor and buffy coat at T1 but not in buffy coat DNA of normal persons/parental/sibling buffy coat DNA, to determine loci and mutations that are common between response groups (i.e common between responders, common between partial responders or common between non-responders) by RECIST criteria or any other method to classify/categorize response to therapy (Example 6).

Creating a database of the specific CNV/SNP/MNP/Indel loci and CNV/SNP/MNP/Indel mutations which are commonly present in the DNA of buffy coat and tumor at T1 that are common across patients in each response group but not shared across patients in different response groups but absent in DNA of normal persons/parental/sibling buffy coat DNA. Using of this database so created to screen for identifying mutations and pathways that are characteristic and predict response of each response group thus identifying the biological processes that are causal to the phenotype exhibited. Using such mutations and pathways to develop drugs and therapies to such response category specific mutations and pathways. The screening for such loci and mutations may be done in the tumor and/or buffy coat at T1. When buffy coat is sampled serially as treatment progresses, the mutation list can be used to monitor response to therapy and quantify residual disease.

The ability to predict which patient will respond to a particular therapy may be used at a. the individual patient level or b. even in planning Phase II/III clinical trials (based on analysis of phase I/II trials). The former (a) will aid optimal therapy for an individual patient and the latter (b) will aid better patient selection, lower cost, substantially reduce time for recruitment and conduct of phase II and III clinical trials or even in repurposing of drugs and novel combinations of known and new drugs.

In yet another embodiment the invention, the invention provides a method to determine mutations of indicative of dose and toxicity of therapies, wherein, the method comprises steps of:
1. Obtaining DNA from buffy coat and tumor of cancer patients belonging to groups that are classified as low dose responder (effectiveness of the drug) or those that exhibit toxicity at recommended dose and those that exhibit toxicity at lower than recommended dose at T1 before starting any treatment, and identifying CNV/SNP/MNP/Indel loci and CNV/SNP/MNP/Indel mutations in DNA of tumor and buffy coat of such groups of patients at T1.
2. obtaining DNA from buffy coat normal cancer-free persons/parental/sibling DNA, identifying CNV/SNP/MNP/Indel loci and CNV/SNP/MNP/Indel mutations in DNA of buffy coat of normal cancer-free persons;
3. determining CNV/SNP/MNP/Indel loci and CNV/SNP/MNP/Indel mutations which are present commonly in the DNA of tumor and buffy coat of these defined groups of cancer patients at T1 but absent in buffy coat DNA of normal persons/parental/sibling DNA and thus to establish a panel loci and mutations that are common between groups of patients that are low dose responders, or groups of patients who exhibit toxicity at lower than recommended dose or groups of patients who exhibit toxicity at recommended or higher doses of a given drug or combination of drugs The toxicity mentioned may apply to any organ eg. bone marrow, liver, kidney, lung, brain, skin, etc. This panel may be used to screen patients and provide low dose responders with longer durations of therapy and avoid doses that cause toxicity at recommended doses in defined individuals or choose individuals who may tolerate a higher than conventional dose. This method may also be used to choose alternative therapies in the event one drug or a combination of drugs causes toxicity where the oncologist may choose an alternative regimen/protocol or develop novel protocols.

Creating a database of the specific CNV/SNP/MNP/Indel loci and CNV/SNP/MNP/Indel mutations which are only present in the DNA of buffy coat and tumor at T1 (but not in normal persons/parental/sibling buffy coat DNA) that are common across patients in each dose response group but not shared across patients in different dose response groups. Using this database so created to screen for identifying mutations and pathways that are characteristic of each response group thus identifying the biological processes that are causal to the phenotype exhibited. Using such mutations and pathways to titrate dose of treatment or to avoid drugs if severe toxicity is seen in some groups of patients or to use these mutations to choose other drugs for the given cancer. This method can be applied for combinations of drugs as well as most often cancer patients are treated with more than one anti-cancer therapy.

Based on such algorithms and panels so developed oncologists may develop treatment regimens including combinations of drugs for new indications of known drugs and combinations thereof.

In all of the above examples, developing a panel of such loci and mutations that are indicative of each embodiment/clinical situation and further developing an amplicon based or other method-based sequencing to identify patients belonging to a specified group as per embodiment above. While these examples given are for exome data, similar logic can be applied to other omics data as well.

In yet another embodiment the invention, the invention provides a method to determine mutations of indicative of early or late recurrence of a given cancer, wherein, the method comprises replicating the method described for responder vs non responders while substituting them with early and late recurrence patients as samples.

In another embodiment, the present invention provides a method to determine various mutations in tumor tissue when the tumor tissue is not available or cannot be obtained, wherein, the blood from the patient is used to obtain buffy coat cells and DNA therefrom is subjected to exome sequencing (or other omics analysis). In order to distinguish gene loci and mutations that are shared/common between buffy coat and the tumor of the patient (thus arriving at gene/mutation list for various embodiments above, the true germline gene loci and mutations (variations) are deduced by sequencing the buffy coat of the patients' parents or biological sibling/alternative source of germline DNA from the patient eg. Buccal swab DNA and defining the variations by comparing with a reference genome. The latter (parental/sibling/alternative germ line) are then subtracted from the variations found in the patients' buffy coat, to arrive at variations shared between the patients' buffy coat and patients' tumor. This method can be applied to all the embodiments described above.

In the various examples above, normal reference genome is preferably that which belongs to the same ethnic/racial group and more preferably that which is derived from both the parental (mother and father of the patient) DNA or biological sibling.

### Definitions

Buffy coat—As used herein, the term “buffy coat” is the fraction of an anti-coagulated blood sample that contains most of the white blood cells and platelets following (density gradient) centrifugation after removal of RBC and plasma/serum.

Indel(s)—As used herein, the term “Indel(s)” or Indel mutations refer to insertion and/or deletion of nucleotides (less than 1 KB) into genomic DNA which result in mutation.

Locus/Loci—As used herein, the term “locus” or its plural form “loci” is a location or address of any length of nucleotides (or base pairs) which may have a variation across genomes.

Mutation—As used herein, the term “Mutation” refer to changes in the genetic sequence of a gene or any DNA segment or fragment as compared to a “normal” person.

SNP(s)/MNP's—As used herein, the term SNP stands for Single Nucleotide Polymorphism and MNP stands for Multiple nucleotide polymorphism

SNP mutations—As used herein, the term “SNP mutations” refers to single-nucleotide substitutions of one base for another which are mutations.

CNV—As used herein, the term SCNV stands for Copy number variation which indicates change in copies of a specified gene as compared to the reference genome.

T1—As used herein, the term “T1” refers to patient with primary cancer/tumor who has not started with any kind of cancer therapy.

T2/3—As used herein, the term “T2” refers to patient with secondary cancer/tumor wherein the patient shows recurrence of tumor at primary tumor tissue/organ or spread of cancer to new tissues/organs (metastasized cancer) after treatment/therapy towards primary cancer/tumor. T3 occurs after T2.

Tn—As used herein, the term “Tn” refers to patient with secondary cancer/tumor wherein the patient shows recurrence of tumor at primary tumor tissue/organ or spread of cancer to new tissues/organs (metastasized cancer) after treatment/therapy for the nth time towards cancer/tumor.

NGS: Next generation sequencing (sequencing of DNA or RNA other nucleic acids and their modifications eg methylome.

### EXAMPLES

### Example 1

### Collection and Sampling, Extraction, and Sequencing of DNA Samples

Blood/tumor collection is done as per standard procedures. DNA is extracted, quality checked and subjected to next generation sequencing using industry standard methods for all the examples below.

### Example 2

### Processing of Data for Identification of Loci and Mutations

Raw Data from NGS studies in FASTq/or equivalent→Raw Data Quality Control→Trim Galore/equivalent→Alignment to GrCH38/parental/reference genome/other acceptable equivalent using BWA MEM→.bam file output as input to FreeBayes/GATK Software/other software→Variation calls and effect report as .vcf format. Use relevant pipeline for CNV determination eg.

### Canvas/Conifer etc

1. Unfiltered VCF files (Can be buffy coat (patient/normal) or Tumor)—paired per patient per particular tumor and tumor state (primary/recurrent/resistant/metastatic/responsive/non responsive etc.).
2. Filter VCF by
a. Quality>=30
b. Raw Read Depth>=20
3. Remove variants that have less than 5 reads supporting it.
4. List these variants along with the following annotation: Chromosome, Position, Ref allele, Variant Allele, Variant type (CNV/SNP/MNP/INDEL), Total read depth at that position, Read depth distribution of variant at each position, Variant Allele frequency at each position, gene name. (So that we have the main VCF file annotated)
5. With the VCF files generated in step 4:
a. Generate a list of mutated positions that are commonly mutated across specific sets of VCF files. (eg. Common across buffy coat; common across tumors for each cancer/response category/dose/toxicity category etc and common across buffy coat and tumor of patients with a particular cancer/cancer stage/response group but not present in normal person's buffy coat/reference genome/parental/sibling/alternate germline genome)
b. Generate a list of mutated positions that are uniquely mutated in a selected VCF file (vs another VCF file) (Seen in single patients' cancer and not in their buffy coat or vice versa). This is to define patient specific mutations.
c. Generate a list of mutated positions that are common to a particular set of VCF files but not mutated in another set of VCF files (Common to cancer buffy coat but not seen in normal buffy coat; Common to cancer (tumor or/and buffy coat DNA but not seen in normal buffy coat, common across cancer patients buffy coat and cancer (with various characteristics described above, but not in normal or parental/sibling/alternative germline DNA/buffy coat)

### Example 3

Determining Susceptibility to/Presence of Cancer

DNA of buffy coat of fourteen cancer patients and buffy coat of two cancer-free subjects were sequenced and subjected to identify SNP/INDEL burden based on the filter criteria explained in Example 2. The total SNP/INDEL burden in buffy coat of cancer-free and cancer patients was compared.

The normal cancer free patients and cancer patients were of same ethnicity.

Table 1 provides the SNP and INDEL mutation burden in DNA of buffy coat of cancer-free subjects (N1 and N2) and solid cancer patients (C1-C14).

TABLE 1

| | VARIANTS | INDEL |
|---|---|---|
| | Q > 29 | Q > 29 |
| Subject | Depth > 19 | DEPTH >= 5 |
| N1 | 37793 | 1490 |
| N2 | 36671 | 1473 |
| C1 | 44082 | 2118 |
| C2 | 43196 | 2194 |
| C3 | 43923 | 2142 |
| C4 | 44156 | 2152 |
| C5 | 54882 | 3523 |
| C6 | 55523 | 3526 |
| C7 | 54528 | 3433 |
| C8 | 54419 | 3493 |
| C9 | 54208 | 3485 |
| C10 | 54967 | 3554 |
| C11 | 55375 | 3354 |
| C12 | 55781 | 3549 |
| C13 | 55431 | 3445 |
| C14 | 54789 | 3512 |

Average variant count (Normal = 37232, Cancer = 51804) Average indel count Normal = 1481, Cancer = 3105)

As evident from the data in the Table 1 the overall Indel/SNP burden in DNA of cancer patient's buffy coat is higher than in normal person's buffy coat. The SNP burden in DNA of buffy coat is at least 1.39 times more, and Indel burden is at least 2 times more in cancer patients than cancer-free subjects.

Hence, if the buffy coat of a subject who is suspected to have a solid cancer or is being screened for solid cancer, is sequenced for Indel/SNP burden in DNA of buffy coat and compared with that of DNA of buffy coat of cancer-free individual, and the overall burden is more than that of cancer free individual it can be concluded that the subject is susceptible or has solid cancer and can be advised to further cancer tests for early diagnosis.

### Example 4

Determining CNV/SNP/MNP/Indel Loci and Mutations Predicting Susceptibility to/Presence of Cancer(s)

To determine susceptibility of person to cancer, it is important to identify markers which are specific to cancers. The method below provides criteria to identify SNP/Indel loci and Mutations which are specific to oral cancer. The SNP/Indel loci and Mutations in DNA of tumor and buffy coat of cancer patients and DNA of buffy coat of cancer-free subjects were determined.

The SNP/Indel loci and Mutations common to DNA of tumor and buffy coat of oral cancer patients but which were absent in DNA of buffy coat of the cancer-free subjects were identified. The normal cancer free patients and oral cancer patients were of same ethnicity

About 238 INDEL Loci were identified to be common to tumor and cancer-buffy coat DNA but not seen in cancer-free subjects; and around 195 INDELs were identified to be common to tumor and cancer-buffy coat but not seen in cancer-free subjects. Similar lists can be computed for other types of mutations such as SNP, MNP's etc. Table 2 provides a list of 10 such representative INDEL Loci and INDELS. Eventual utility of this approach is for use as a blood test for screening for cancer.

TABLE 2

| INDEL LOCI | | EXACT INDEL | |
|---|---|---|---|
| Loci-1 | chr7- | INDEL-1 | chr12-76424937- |
| | 23195555 | | ttgctgctgctgctg |
| Loci-2 | chr17- | INDEL-2 | chr16-8904877- |
| | 73619955 | | TATGATG |
| Loci-3 | chr4- | INDEL-3 | chr6-31514153- |
| | 183268148 | | GCTGTCT |
| Loci-4 | chr15- | INDEL-4 | chr3-105412286- |
| | 98995052 | | CTTT |
| Loci-5 | chr2- | INDEL-5 | chr12-122064773- |
| | 150435889 | | cccgccaccgcc |
| Loci-6 | chr21- | INDEL-6 | chr6-312029-TAA |
| | 46924425 | | |
| Loci-7 | chr1- | INDEL-7 | chr11-100827326- |
| | 78430689 | | aaat |
| Loci-8 | chr1- | INDEL-8 | chr8-31005017-TC |
| | 203137784 | | |
| Loci-9 | chr8- | INDEL-9 | chr21-32201969- |
| | 38006330 | | GA |
| Loci-10 | chr16- | INDEL-10 | chr17-27246368- |
| | 5122072 | | CCTCCCCTCCTTGATC |
| | | | CTAACATTCTCCCCT |

### Example 5

Determining CNV/SNP/MNP/Indel Loci and Mutations that Indicate/Predict Recurrence of Cancer

DNA was extracted from buffy coat of cancer patients (at T 1 time point) and recurrent solid cancer (T1, T2, T3 time points) and from buffy coat of cancer free subjects. The process described above was followed to determine patient/normal buffy coat and tumor specific indels.

Around 436 INDEL Loci were identified which were unique to DNA of cancer patient-buffy coat at T1 solid cancer patients but absent in buffy coat DNA of cancer-free subjects; and around 388 INDELs were identified which were unique to DNA of cancer patient-buffy coat of T1 solid cancer patients but absent in DNA of buffy coat of cancer-free subjects. Thus, presence of these mutations in buffy coat at T1 stage indicates indicate presence/recurrence of cancer and possible development of these mutations at T2 and later stages. Table 3 provides 10 representatives of such INDEL Loci and INDEL mutations.

TABLE 3

| INDEL LOCI | | EXACT INDEL | |
|---|---|---|---|
| Loci-1 | chr14-50949196 | INDEL-1 | chr17-39991867-ATATC |
| Loci-2 | chr12-82828426 | INDEL-2 | chr3-112301458- |
| | | | CCTTAGTAATCTT |
| Loci-3 | chr11-108098459 | INDEL-3 | chrX-153149708-GC |
| Loci-4 | chr7-75616817 | INDEL-4 | chr8-145168298-TGGGGG |
| Loci-5 | chr20-36488508 | INDEL-5 | chr14-71216863-CAT |
| Loci-6 | chr3-197500394 | INDEL-6 | chr4-1737404-CCT |
| Loci-7 | chr14-22998619 | INDEL-7 | chr15-34118994-CAG |
| Loci-8 | chrX-47487912 | INDEL-8 | chr19-47933190-AGGGG |
| Loci-9 | chr4-1737404 | INDEL-9 | chr14-96671207- |
| | | | Gggggggacggtggggac |
| Loci-10 | chr10-98979111 | INDEL- | chr4-155244401- |
| | | 10 | Ttttgtttgtttgtttgtttgttt |

These mutations are probably indicative of presence of cancer, recurrence of cancer or metastasis. A database of such mutations will enable screening patients who are more susceptible to metastasis or recurrence of cancer. Moreover, this screening requires only testing of DNA of buffy coat derived from simple blood sample and does not require biopsy.

Around 499 INDEL Loci were identified which were unique to DNA of tumor T2 or T3 solid cancer patients but absent in DNA of cancer—of T1 cancer patients but present in the DNA of buffy coat at T1; and around 454 INDELs were identified which were unique to DNA of cancer—at T2 or T3 solid cancer patients but absent in DNA of cancer—of T1 cancer patients but present in the DNA of buffy coat of T1. Table 4 provides 10 representatives of such INDEL Loci and INDEL mutations.

TABLE 4

| INDEL LOCI | | EXACT INDEL | |
|---|---|---|---|
| Loci-1 | chr14-50949196 | INDEL-1 | chr21-38599934-TGG |
| Loci-2 | chr12-82828426 | INDEL-2 | chr17-39991867-ATATC |
| Loci-3 | chr7-75616817 | INDEL-3 | chr3-112301458- |
| | | | CCTTAGTAATCTT |
| Loci-4 | chr20-36488508 | INDEL-4 | chrX-153149708-GC |
| Loci-5 | chr12-10786757 | INDEL-5 | chr8-145168298-TGGGGG |
| Loci-6 | chr3-53324819 | INDEL-6 | chr14-71216863-CAT |
| Loci-7 | chr3-197500394 | INDEL-7 | chr10-118383463-AGG |
| Loci-8 | chr14-22998619 | INDEL-8 | chr4-1737404-CCT |
| Loci-9 | chrX-47487912 | INDEL-9 | chr15-34118994-CAG |
| Loci-10 | chr4-1737404 | INDEL- | chr19-47933190-AGGGG |
| | | 10 | |

Around 409 INDEL Loci were identified which were unique to tumor DNA of T2 or T3 solid cancer patients but absent in DNA of tumor of T1 cancer patients and DNA of buffy coat of cancer-free patients, and present in the buffy coat of T1 cancer patients; and around 368 INDELs were identified which were unique to DNA of tumor—of T2 or T3 solid cancer patients but absent in DNA of tumor of T1 cancer patients and DNA of buffy coat of cancer-free patients, and present in the buffy coat of T1 cancer patients. Table 5 provides 10 representatives of such INDEL Loci and INDEL mutations.

TABLE 5

| INDEL LOCI | | EXACT INDEL | |
|---|---|---|---|
| Loci-1 | chr14-50949196 | INDEL-1 | chr17-39991867-ATATC |
| Loci-2 | chr12-82828426 | INDEL-2 | chr3-112301458- |
| | | | CCTTAGTAATCTT |
| Loci-3 | chr7-75616817 | INDEL-3 | chrX-153149708-GC |
| Loci-4 | chr20-36488508 | INDEL-4 | chr8-145168298-TGGGGG |
| Loci-5 | chr3-197500394 | INDEL-5 | chr14-71216863-CAT |
| Loci-6 | chr14-22998619 | INDEL-6 | chr4-1737404-CCT |
| Loci-7 | chrX-47487912 | INDEL-7 | chr15-34118994-CAG |
| Loci-8 | chr4-1737404 | INDEL-8 | chr19-47933190-AGGGG |
| Loci-9 | chr10-98979111 | INDEL-9 | chr14-96671207- |
| | | | Gggtggggacggtggggac |
| Loci-10 | chr15-102029685 | INDEL-10 | chr4-155244401- |
| | | | Ttttgtttgtttgtttgtttgttt |

These are probably mutations indicative of directed mutagenesis driven by DNA of Buffy coat, i.e. the DNA of buffy coat of T1 cancer patients can be used to identify recurrence of tumor. These indicate mutations as the cancer evolves from T1 to T2 and T3. Eventually utility of this approach for use as a blood test.

In the examples above INDEL examples are provided. Similar lists can be compiled for other mutations such as CNV, SNP, MNP etc.

### Example 6

Determining SNP/Indel Loci and Mutations/Genes that are Indicative of Responsiveness of Cancer Therapy/Treatment—Including Determining Pathways Responsible for Tumor Behavior; Utility in Cancer Clinical Trials (to Choose Responders in pH II and pH III Trials; Rational Choice of Agents Based on Pathways Involved and Responsivity to Particular Agents.

13 patients who were diagnosed to have melanoma by histopathology were included in the study. Their tumor and blood buffy coat samples were obtained prior to start of anti PDL 1 antibody therapy. At the conclusion of the therapy cycles, the patients were determined to have responded or otherwise to the therapy and categorized into three groups—complete responder (3 patients), partial responder (5 patients) and progressive disease—5 patients (non-responder) according to RECIST criteria. Their buffy coat DNA and tumor DNA were subjected to exome sequencing using standard methods. SNP/MNP/INDELS common to buffy coat and tumor in each group were determined by analysis and comparison of VCF files. Table 6 provides a representative list of loci that were mutated across members of a particular response group (selected based on their presence in both buffy coat DNA and tumor of the patients in the group). This database of loci can be used to screen the buffy coat of melanoma patients who are slated to undergo anti PDL 1 therapy and a determination/prediction can be made of their response status. Commonality of mutations (in buffy coat and tumor) across patients of a particular response/toxicity group may also be generated using the same logic. Such markers may also be used to monitor progress of therapy, minimal residual disease; see example 7.7 for more data).

TABLE 6

| Complete | Partial | Non |
|---|---|---|
| Response Loci | Response Loci | Responder Loci |
| (Chromosome | (Chromosome | (Chromosome |
| number and | number and | number and |
| position | position | position |
| Chr7_20739023 | chr16_4115431 | chr1_1047614 |
| Chr2_219218557 | chr3_186619924 | chr2_130508957 |
| Chr2_219218731 | chr3_186620593 | chr3_142449489 |
| Chr6_43432099 | chr3_186620636 | chr4_107720144 |
| Chr5_179207618 | chr6_151348740 | chr5_14419918 |
| Chr9_18776842 | chr6_151349037 | chr6_12294025 |
| Chr7_45714595 | chr6_151349762 | chr7_92501975 |
| Chr1_111499761 | chr1_19282760 | chr8_102560764 |
| Chr1_111500010 | chr1_19284747 | chr8_27454186 |
| Chr5_132879785 | chr7_123616373 | chr9_70282518 |

Responsible pathways for resistance thus determined may be used to choose alternative or add on therapies for the non-responder and partial responder groups.

Such an approach may be used to determine response status of any cancer to any therapy (single or combination), including chemotherapy, radiation therapy, targeted therapy (small or large molecule);

Such an approach when applied to Phase II clinical studies, the responder signatures may be used to recruit patients into the Phase III clinical trial thus reducing the time, cost and effort of Phase III trials.

Such an approach may be utilized for determining novel applications for known drugs as well.

While certain exemplary embodiments have been described and shown in the accompanying tables, it is to be understood that such embodiments are merely illustrative of, and not restrictive on, the broad invention, and that this invention not be limited to the specific mutations and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention.

### Example 7

### Screening Test of the Subjects Having Cancer:

Numbers indicate unique mutations found in each instance. Nos. 2-6 below can be used for screening for primary and secondary/recurrent cancers by a blood test. No 7 is for response to therapy prediction and also for screening for melanoma (primary and recurrent/metastatic). Response to therapy prediction when applied to Ph II clinical trial setting can then be used to recruit particular (responders only) patients to Ph III clinical trials thus enhancing response rate in Ph III trials. Subsequently, this gene mutation set can then be utilized as a companion diagnostic for the new/modified therapy along with the marketed drug.
1. Normal:
   10 normal subjects of Indian origin. Buffy coat DNA was sequenced;
   Common mutations across all 10 subjects=6466.
2. Lung Cancer (sensitive):
   10 patients—buffy coat and tumor sequenced;
   Common in all 10 tumor (T)=5419
   Common in all 10 blood (B)=7428
   Common in all ten B and T=4571
   Compared all B+T 4571 with normal 6466
   Unique to cancer B+T=2130−can be used to screen for lung cancer
   using a blood sample.
   Common with normal=2411
   Sample of Unique mutations common to buffy coat and tumor in lung cancer (out of 2130 mutations); only deletions are shown, list includes deletions, insertions and SNV's

| VarType | GeneSymbol | Variant Loci | Variant_Allele |
|---|---|---|---|
| Deletion | ACADSB [+] | chr10-123053169 | AT-A |
| Deletion | ALDH3A2 [+] | chr17-19657698 | GACTGAATT-G |
| Deletion | ATG13 [+] | chr11-46672763 | GTC-G |
| Deletion | ATL3 [−] | chr11-63646490 | AAAAT-A |
| Deletion | CLCNKB [+] | chr1-16046704 | CCTT-C |
| Deletion | CLCNKB [+] | chr1-16049743 | TC-T |
| Deletion | CNTLN [+] | chr9-17457514 | TA-T |
| Deletion | COL18A1 [+] | chr21-45504511 | CGGCCCCCCA-C |
| Deletion | CPT1A [−] | chr11-68799179 | GAAA-G |
| Deletion | DLAT [+] | chr11-112045277 | AG-A |

3. Breast Cancer:
   10 patients—buffy coat and tumor sequenced;
   Common in all 10 tumor=4088
   Common in all 10 blood=5696
   Common in all ten B and T=3561
   Compared all B+T 3561 with normal 6466
   Unique to cancer B+T=1798 can be used to screen for breast cancer using a blood sample.
   Common with normal=1763
   Sample of Unique mutations common to buffy coat and tumor in breast cancer (out of 1798 mutations) only deletions are shown, list includes deletions, insertions and SNV's

| VarType | GeneSymbol | Variant Loci | Variant Allele |
|---|---|---|---|
| Deletion | ADAM17 [−] | chr2-9490115 | AT-A |
| Deletion | ANO5 [+] | chr11-22276060 | AT-A |
| Deletion | ARAP2 [−] | chr4-36128753 | TA-T |
| Deletion | ATG13 [+] | chr11-46672763 | GTC-G |
| Deletion | ATL3 [−] | chr11-63646490 | AAAAT-A |
| Deletion | B3GLCT [+] | chr13-31215032 | CT-C |
| Deletion | BCKDHB [+] | chr6-80343999 | TTTG-T |
| Deletion | C15orf40 [−] | chr15-83004727 | TA-T |
| Deletion | CACNG7 [+] | chr19-53942340 | AG-A |
| Deletion | CATSPERE +] | chr1-244639903 | CTT-C |

4. Pancreatic Cancer:
   10 patients—buffy coat and tumor sequenced;
   Common in all 10 tumor=7261
   Common in all 10 blood=6148
   Common in all ten B and T=5216
   Compared all B+T 5216 with normal 6466
   Unique to cancer B+T=2898 can be used to screen for pancreatic cancer using a blood sample.
   Common with normal=2318
   Sample of Unique mutations common to buffy coat and tumor in pancreatic cancer (out of 2898 mutations) only deletions are shown, list includes deletions, insertions and SNV's

| VarType | GeneSymbol | Variant_Loci | Variant_Allele |
|---|---|---|---|
| Deletion | AARSD1 [−] | chr17-42964274 | CAGCCCT-C |
| Deletion | AKAP1 [+] | chr17-57105450 | CT-C |
| Deletion | ANK2 [+] | chr4-113365201 | ATG-A |
| Deletion | ATP1A2 [+] | chr1-160120891 | CCCTT-C |
| Deletion | C1QL2 [−] | chr2-119157556 | CG-C |
| Deletion | CCT5 [+] | chr5-10263341 | GA-G |
| Deletion | CCT5 [+] | chr5-10263349 | GGGA-G |
| Deletion | CD4 [+] | chr12-6800214 | TG-T |
| Deletion | CDCA5 [−] | chr11-65083981 | CT-C |
| Deletion | CENPJ [−] | chr13-24884094 | AGTT-A |

5. Lung Cancer (resistant):
   10 patients—buffy coat and tumor sequenced;
   Common in all 10 tumor=5639
   Common in all 10 blood=7428
   Common in all ten B and T=5167
   Compared all B+T 5167 with normal 6466
   Unique to cancer B+T=2501 can be used to screen for (resistant) lung
   cancer using a blood sample.
   Common with normal=2666
   Sample of Unique mutations common to buffy coat and tumor in lung cancer (resistant) (out of 2501 mutations) only deletions are shown, list includes deletions, insertions and SNV's

| | | | Variant_ |
|---|---|---|---|
| VarType | GeneSymbol | Variant_Loci | Allele |
| Deletion | ABCB5 [+] | chr7-20700151 | AT-A |
| Deletion | ACADSB [+] | chr10-123053169 | AT-A |
| Deletion | ADGRF5 [−] | chr6-46867169 | TG-T |
| Deletion | AFDN [+] | chr6-167947824 | CT-C |
| Deletion | ALDH3A2 [+] | chr17-19657698 | GACTGAATT-G |
| Deletion | ARAP2 [−] | chr4-36128753 | TA-T |
| Deletion | ATG13 [+] | chr11-46672763 | GTC-G |
| Deletion | ATL3 [−] | chr11-63646490 | AAAAT-A |
| Deletion | CDCA5 [−] | chr11-65083981 | CT-C |
| Deletion | CEP290 [−] | chr12-88084539 | TA-T |

6. Comparison across Lung, Breast and Pancreatic cancers:
   Lung=2130 vs Breast=1798 vs Pancreas=2898 (from above lists) Common across Lung, Breast and Pancreas=165—can be used to screen for lung, breast and pancreatic cancers using a blood sample
   Common across Lung and Breast=329 can be used to screen for lung and breast cancers using a blood sample
   Common across Lung and Pancreas=903 can be used to screen for lung and pancreatic cancers using a blood sample
   Common across Breast and Pancreas=327 can be used to screen for pancreatic and breast cancers using a blood sample
   Sample of Unique mutations common to buffy coat and tumor in lung, breast and pancreatic cancer (out of 165 mutations)

| | | | Variant_ |
|---|---|---|---|
| VarType | GeneSymbol | Variant_Loci | Allele |
| Deletion | DLAT [+] | chr11-112045277 | AG-A |
| Deletion | OPTN [+] | chr10-13110504 | GT-G |
| Deletion | SFMBT1 [−] | chr3-52925997 | GC-G |
| Deletion | TTPA [−] | chr8-63066099 | TA-T |
| Deletion | USH2A [−] | chr1-215680406 | GT-G |
| Deletion | WRN [+] | chr8-31100837 | CT-C |
| Insertion | HSP90AA1 -1 | chr14-102085458 | T-TTAA |
| Insertion | PCLO [−] | chr7-82952172 | T-TTCA |
| Insertion | TAF1D [−] | chr11-93741404 | T-TGCGTGA |
| Insertion | TMEM87A [−] | chr15-42231143 | T-TC |
| SNV | AARSD1 [−] | chr17-42964217 | G-A |

7. Melanoma treated with anti PDL 1 antibody:
   13 patients—5 partial responders, 5 non responders, 3 complete responders
   Common to all T=8089
   Common to all B=9345
   Common to all T+B=7362−this can be used for screening for
   melanoma primary and recurrence using a blood sample
   Partial responders:
   Common to all B=15482
   Common to all T=14946
   Common to all B and all T=13449
   Non responders:
   Common to all B=16494
   Common to all T=14466
   Common to all B and all T=13449
   Complete responders:
   Common to all B=23630
   Common to all T=20552
   Common to all B and all T=19376
   Comparing between 3 groups:
   Unique to partial responders=2362
   Unique to non-responders=2218
   Unique to complete responders=6949
   A screen comprising of 2362+2218 and 6949 mutations can be used to determine response status of patients slated to undergo anti PDL 1 therapy for melanoma.
   Sample of Unique mutations common to buffy coat and tumor in melanoma complete responders (out of 6949 mutations) only deletions are shown, list includes deletions, insertions and SNV's

| | | Variant_ | Variant_ |
|---|---|---|---|
| VarType | GeneSymbol | Loci | Allele |
| Deletion | A1BG [−] | chr19-58346865 | GC-G |
| Deletion | ABCA4 [−] | chr1-94011435 | CT-C |
| Deletion | ABCC4 [−] | chr13-95020996 | AT-A |
| Deletion | ABCC4 [−] | chr13-95062894 | AAAAG-A |
| Deletion | ABHD10 [+] | chr3-111991354 | AT-A |
| Deletion | ABHD16A [−] | chr6-31701372 | TAC-T |
| Deletion | ABI3BP [−] | chr3-100875482 | CGGCATA-C |
| Deletion | ABR [−] | chr17-1005655 | AAG-A |
| Deletion | ABRAXAS1 [−] | chr4-83459882 | CA-C |
| Deletion | ACSL3 [+] | chr2-222916299 | AT-A |

Sample of Unique mutations common to buffy coat and tumor in melanoma partial responders (out of 2362 mutations) only deletions are shown, list includes deletions, insertions and SNV's

| | | | Variant_ |
|---|---|---|---|
| VarType | GeneSymbol | Variant_Loci | Allele |
| Deletion | ACSM6 [+] | chr10-95207197 | GT-G |
| Deletion | ADGRG4 [+] | chrx-136392285 | AGAT-A |
| Deletion | APOL4 [−] | chr22-36191797 | ACT-A |
| Deletion | ARHGAP26 | chr5-143224107 | CT-C |
| Deletion | ARL4C [−] | chr2-234495139 | CT-C |
| Deletion | ARRDC4 [+] | chr15-97969392 | CT-C |
| Deletion | BDKRB2 [+] | chr14-96204870 | GGGTGG |
| | | | GGAC-G |
| Deletion | BLMH [−] | chr17-30248432 | GCA-G |
| Deletion | CATSPERE [+] | chr1-244639903 | CTT-C |
| Deletion | CCDC25 [−] | chr8-27740525 | GA-G |

Sample of Unique mutations common to buffy coat and tumor in melanoma non responders (out of 2218 mutations) only deletions are shown, list includes deletions, insertions and SNV's

| | | | Variant_ |
|---|---|---|---|
| VarType | GeneSymbol | Variant_Loci | Allele |
| Deletion | ADGRL3 [+] | chr4-61909761 | ATG-A |
| Deletion | AP1S3 [−] | chr2-223758143 | TA-T |
| Deletion | APOBEC3H [+] | chr22-39100317 | TAAC-T |
| Deletion | ARMH4 [−] | chr14-58012155 | GA-G |
| Deletion | ASCL1 [+] | chr12-102960408 | CT-C |
| Deletion | AXL [+] | chr19-41253726 | ACCC-A |
| Deletion | BASP1 [+] | chr5-17275944 | CCT-C |
| Deletion | BTN1A1 [+] | chr6-26501243 | AG-A |
| Deletion | BUD23 [+] | chr7-73697727 | TG-T |
| Deletion | CASD1 [+] | chr7-94528273 | CT-C |

1. A method for detecting cancer susceptibility, early detection and predicting cancer behaviour by using specific markers and parameters wherein the method comprises of:
screening persons with high risk of getting/having a type of cancer; identifying CNV/SNP/MNP/Indel loci and/or CNV/SNP/MNP/Indel mutations specific to a cancer and common in the buffy coat and tumor of cancer patients;
subjecting the buffy coat and tumour of cancer patients to the step of identification of common loci and/or Indel/SNP/CNV/MNP mutations in chromosomes which indicate recurrence of cancer and/or metastasis;
predicting mutations that will occur when metastasis or recurrence of cancer/tumor occurs by identifying CNV/Indel/SNP/MNP loci and/or Indel/SNP/CNV/MNP mutations in chromosomes in buffy coat and tumor by analysis of defined and timed samples of buffy coat, primary, secondary/recurrent tumor;
determining loci and mutations indicative of the tumor behaviour, selected from responsiveness to treatment, toxicity of therapies, dose optimization, minimal residual disease; and indicative of progression of disease, timing of recurrence and determining pathways responsible for various behaviours.

2. The method as claimed in claim 1, wherein the step of screening people for cancer or susceptibility to cancer comprising of:
determining Indel and/or CNV/SNP/MNP burden in DNA of buffy coat of known cancer patients/and of a normal cancer-free persons with respect to the reference genome, and determining the ratio of Indel and/or CNV/SNP/MNP burden in DNA of buffy coat of the persons with cancer and that of normal cancer-free persons;
wherein, if the ratio of Indel and/or CNV/SNP/MNP burden of the subject is at least 1.39 times for SNP/MNP/CNV and indel burden is at least 2 times than that of normal cancer free subjects then concluding that a subject is susceptible to a cancer or already has cancer.

3. The method as claimed in claim 1, wherein the step of identifying CNV/SNP/MNP/Indel loci and/or CNV/SNP/MNP/Indel mutations specific to a cancer in the buffy coat and tumour of cancer patients comprises of; obtaining DNA from buffy coat, and malignant tumors/cancer cells of tissues/organs/bone marrow of a patient with a specific cancer (primary or recurrent or metastatic cancer) and sequencing the DNA of both buffy coat and tumor tissue and establishing the CNV/SNP/MNP/INDEL loci and mutations present and common in both samples;
obtaining DNA from buffy coat of a normal cancer-free persons/parental/sibling buffy coat DNA sequencing the DNA using standard NGS methods and establishing CNV/SNP/MNPs/INDEL loci and mutations in such cancer free individuals with respect to a reference genome;
determining the CNV/SNP/MNPs/INDEL loci and mutations which are present in the DNA of both buffy coat and tumors of tissues/organs of the patient with specific cancer, but absent from the DNA of buffy coat of normal cancer-free person/reference genome/parental/sibling buffy coat genome, wherein, said CNV/SNP/MNPs/INDEL loci and mutations indicative of cancer-specific mutations; and creating a database of said cancer specific CNV/SNP/MNPs/INDEL loci and mutations; and using this gene mutation list/database to screen for mutations in DNA of buffy coat of subjects desirous of being screened for cancer or screening of patients in remission for evidence of recurrence/metastasis.

4. The method as claimed in claim 1 wherein the step of identification of loci and/or Indel/SNP/CNV/MNP mutations in chromosomes which indicate recurrence of cancer/metastasis comprises the steps of: obtaining DNA from buffy coat and tumor of cancer patients having metastasis or recurrence and identifying CNV/SNP/MNP/Indel loci and CNV/SNP/MNP/Indel mutations common in DNA of buffy coat and secondary or recurrent tumours of cancer patients;
obtaining DNA from buffy coat normal cancer-free person/reference genome/parental/sibling DNA and identifying CNV/SNP/MNP/Indel loci and CNV/SNP/MNP/Indel mutations in DNA of buffy coat of normal cancer free person with respect to the reference genome;
determining CNV/SNP/MNP/Indel loci and CNV/SNP/MNP/Indel mutations which are only present in the DNA of buffy coat of recurrent/metastasis cancer patient, but absent or preferably present in the DNA of primary/recurrent/metastatic tumor of cancer patient and absent in the DNA of buffy coat of normal person/reference genome/parental/sibling DNA, the said loci CNV/SNP/MNP/Indel mutations indicative of recurrence of cancer/tumor and/or metastasis;
creating a database of the specific CNV/SNP/MNP/Indel loci and CNV/SNP/MNP/Indel mutations which are only present in the DNA of buffy coat and may or may not be present in the secondary/recurrent tumor of cancer patient, but absent in the buffy coat of normal persons to serve as markers which indicate primary, metastasis or recurrence of cancer;
and creating a database of said CNV/SNP/MNP/Indel loci and CNV/SNP/MNP/Indel mutations; and using this gene mutation list/database to screen for mutations in DNA of buffy coat of subjects desirous of being screened for cancer or screening of patients in remission for evidence of recurrence/metastasis.

5. The method as claimed in claim 1 wherein the step of identifying CNV/Indel/SNP/MNP loci and/or Indel/SNP/CNV/MNP mutations in chromosomes which predict mutations that occur during metastasis comprises the steps of:
obtaining DNA from buffy coat and tumor when a patient has the primary cancer time point, T1, and identifying CNV/SNPMNP/Indel loci and CNV/SNP/MNP/Indel mutations common in DNA of buffy coat and tumors of cancer patients;
obtaining DNA of buffy coat and tumor of cancer patient with recurrence/metastasis of tumor after initial therapy (time point, T2) or after more than one round of therapy (T3-Tn) leading to remissions (partial response or full response) and identifying CNV/SNP/MNP/Indel loci and CNV/SNP/MNP/Indel mutations in DNA of buffy coat and/or recurrent/metastatic tumor of patient;
determining CNV/SNP/MNP/Indel loci and CNV/SNP/MNP/Indel mutations which are only present in the DNA of buffy coat or tumor of T2 and/or T3 and are present in the DNA of buffy coat at T1, but not in tumor at T1 to establish a loci and mutations indicative/predictive of recurrence or metastasis, and creating a database of said CNV/SNP/MNP/Indel loci and CNV/SNP/MNP/Indel mutations; and/MNP;
determining CNV/SNP/MNP/Indel loci and CNV/SNP/Indel mutations which are only present in the DNA of buffy coat at T1 and/or secondary/recurrent tumor (T2 or T3) of cancer patient, but absent in DNA of tumor of T1 and DNA of buffy coat of normal persons to serve as markers which indicate mutations that may arise at T2 or T3 or Tn as the cancer evolves, and creating a database of said CNV/SNP/MNP/Indel loci and CNV/SNP/MNP/Indel mutations; and
using this gene mutation list/database to screen for mutations in DNA of buffy coat of subjects in whom prediction of mutations that might occur as the cancer progresses is required to be determined for help in designing therapies optimally.

6. The method as claimed in claim 1, wherein the step of determining mutations indicative of responsiveness to treatment comprises the steps of: obtaining DNA from buffy coat and tumor of cancer patients belonging to groups that are classified as responder, partial responder or poor responder/progressive disease (RECIST criteria) or any other mode of classification of response (including by pathology, imaging, biomarker driven and other such methods) at T1 before starting any treatment, and identifying CNV/SNP/MNP/Indel loci and CNV/SNP/MNP/Indel mutations common in DNA of tumor at T1 and in buffy coat of such patients;
obtaining DNA from buffy coat normal cancer-free persons/reference genome/parental/sibling buffy coat genome, identifying CNV/SNP/MNP/Indel loci and CNV/SNPMNP/Indel mutations in DNA of buffy coat of normal cancer-free persons/parental/sibling DNA;
determining CNV/SNP/MNP/Indel loci and CNV/SNP/MNP/Indel mutations which are present commonly in the DNA of tumor and buffy coat at T1 but not in buffy coat DNA of normal persons/parental/sibling buffy coat DNA, to determine loci and mutations that are common between response groups (i.e common between responders, common between partial responders or common between non-responders) by RECIST criteria or any other method to classify/categorize response to therapy; and using this gene mutation list/database to screen for mutations in DNA of buffy coat of subjects for determining if they will respond or otherwise to a particular mode of therapy.

7. The process as claimed in claim 1, wherein the step of determining mutations indicative of dose and toxicity of therapies comprises the steps of:
obtaining DNA from buffy coat and tumor of cancer patients belonging to groups that are classified as low dose responder (effectiveness of the drug) or those that exhibit toxicity at recommended dose and those that exhibit toxicity at lower than recommended dose at T1 before starting any treatment, and identifying CNV/SNP/MNP/Indel loci and CNV/SNP/MNP/Indel mutations in DNA of tumor and buffy coat of such groups of patients at T1;
obtaining DNA from buffy coat normal cancer-free persons/parental/sibling DNA, identifying CNV/SNP/MNP/Indel loci and CNV/SNP/MNP/Indel mutations in DNA of buffy coat of normal cancer-free persons;
determining CNV/SNP/MNP/Indel loci and CNV/SNP/MNP/Indel mutations which are present commonly in the DNA of tumor and buffy coat of these defined groups of cancer patients at T1 but absent in buffy coat DNA of normal persons/parental/sibling DNA and thus to establish a panel loci and mutations that are common between groups of patients that are low dose responders, or groups of patients who exhibit toxicity at lower than recommended dose or groups of patients who exhibit toxicity at recommended or higher doses of a given drug or combination of drugs;
creating a database of the specific CNV/SNP/MNP/Indel loci and CNV/SNP/MNP/Indel mutations which are only present in the DNA of buffy coat and tumor at T1; and
using this gene mutation list/database to screen for mutations in DNA of buffy coat of subjects where prediction of toxicity to particular agents is desirable.

8. Method as claimed in claim 1, wherein the common buffy coat and tumor mutations are defined in the various clinical settings and stages of evolution/progress of cancer/behaviour of cancer, and the blood is used to detect these mutations where biopsy of the tumor is not feasible, difficult or where blood sampling will suffice/is preferred to detect the said cancer and its behaviour.
